(19) United States
(12) Patent Application Publication
(10) Pub. No.: US 2024/0115199 A1
(43) Pub. Date: Apr. 11, 2024

(54) SYSTEMS AND METHODS FOR PREDICTING EFFECTIVENESS OF TREATMENT

(71) Applicant: Magnus Medical, Inc., Burlingame, CA (US)

(72) Inventors: Michael FEYDER, Baltimore, MD (US); Brandon S. BENTZLEY, Half Moon Bay, CA (US); Armani PORTER, Pikesville, MD (US); Eren Kiris, Los Altos Hills, CA (US)

(21) Appl. No.: 18/482,553

(22) Filed: Oct. 6, 2023

Related U.S. Application Data
(60) Provisional application No. 63/378,781, filed on Oct. 7, 2022.

(51) Int. Cl. A61B 5/00; A61B 5/16; A61N 1/36; A61N 2/00; A61N 2/02; G16H 15/00; G16H 20/70

(52) U.S. Cl. CPC A61B 5/4848; A61B 5/165; A61B 5/4088; A61B 5/7264; A61B 5/746; A61N 1/36082; A61N 2/006; A61N 2/02; G16H 15/00; G16H 20/70

(57) ABSTRACT

Described herein are systems and methods for predicting response to treatment for a neurological or psychiatric disorder. The systems may include machine learning and run LDA and/or Lasso Regression methods that use psychometric inventories and other forms of patient data such as patient characteristics, treatment history, clinical history, biometric data, neuroimaging data, or a combination thereof, as inputs to generate a predictive model that separates responders from non-responders. The methods may include prediction of response to initial treatment by obtaining baseline, pretreatment data collected through psychometric inventories as well as other forms of patient data.

### CROSS-REFERENCE TO RELATED APPLICATIONS

This application claims priority to U.S. Provisional Application No. 63/378,781, filed on Oct. 7, 2022, which is hereby incorporated by reference in its entirety.

### TECHNICAL FIELD

This application generally relates to the treatment of a neurological or a psychiatric disorder. More specifically, the application relates to systems and methods for the prediction of response in individuals who will receive treatment for a neurological or a psychiatric disorder. The systems and methods may employ processes that automate the prediction of response. Systems and methods for the prediction of response in individuals who will receive transcranial magnetic stimulation, or neurostimulation using a different treatment modality are also described herein.

### BACKGROUND

Depression, including major depressive disorder (MDD), bipolar disorder (BD), and peripartum depression (PPD), is the leading cause of disability worldwide and is characterized by a high rate of recurrence. However, there are no objective biomarkers, including imaging findings or lab tests, which can help a clinician choose the most effective treatment for a patient experiencing depression, or any other neurological or psychiatric disorder. As a result, clinicians usually choose initial treatment based on factors other than efficacy. For example, in some cases the choice of therapy is elected by a clinician based on the side effect profile a patient is most likely to tolerate. In other cases, the choices are based on the clinician's personal preferences, which may lead a clinician to choose the same initial treatment for most of their patients. This inability to predict a response to treatment may prevent clinicians from choosing well-informed therapeutic interventions for neurological and psychiatric disorders, which can be deleterious for the health of individuals experiencing acute mental illness or a mental health emergency.

Moreover, if a clinician chooses a therapy that is initially successful, patients with a neurological or psychiatric disorder such as treatment-resistant depression (TRD), may have a high relapse rate, with relapse occurring during or after initial TMS treatment. Epidemiological and clinical evidence suggests that major depressive disorder typically follows a recurrent course, with a third to a half of patients relapsing within one year of discontinuation of treatment. A greater number of prior depressive episodes may be generally correlated with a higher probability of a future recurrence. Given this, there is a need to predict those individuals who will respond to treatment, but also those who will relapse prior to the termination of the initial course of treatment.

Further, if a clinician chooses a neurological or psychiatric therapy and an individual does not respond to initial treatment, the individual may respond to subsequent treatments. Given this, there is a need to predict those individuals who will not respond to an initial course of treatment, but to predict those who will respond during subsequent treatments.

Prediction of response would be advantageous to the improvement of the overall disease trajectory as the inability to predict a response to a treatment prevents clinicians from choosing well-informed therapeutic interventions for neurological and psychiatric disorders. Thus, the development of systems and methods that use readily available information, such as biosignals, clinical and treatment history, biometric data (including genomics), and psychometric inventories, for prediction of response may be helpful for the early prediction of response and/or relapse.

Adequate treatment of depression may be hindered by the lack of methods to detect or predict response. Standard antidepressants may be effective for treatment of MDD. However, many TRD patients do not respond to these medications and many patients desire to reduce or end their chronic use of anti-depressants. Acute interventions may also be used to treat depression, as described below; additionally, many patients are in need of acute treatment for depression due to suicidality or hospitalization.

Intravenous (IV) ketamine has been used as a rapid acting antidepressant and has demonstrated efficacy in maintaining an antidepressant effect beyond the acute treatment period. Additionally, long-term maintenance therapy regimens using ketamine have shown promise in prevention of relapse (Sing J B, Fedgchin M, Daly E J, Drevets W C, Adv Pharmacol, 2020, 89:237-259). However, ketamine is a drug of abuse and has serious side effects. Therefore, it may be beneficial to administer the therapy only when needed (on-demand). For example, the ability to trigger therapy after prediction of response would reduce the exposure to ketamine.

Recently, psychedelics have shown some effectiveness as a therapy for TRD (Davis A K, Barrett F S, May D G, et al. JAMA Psychiatry 2021, 78(5):481-489). Currently, the use of psychedelics requires dosing and monitoring by a psychotherapist. This model may be time and therapist intensive (Nutt D, Carhart-Harris R, JAMA Psychiatry, 2020E1-E2). Additionally, psychedelics are scheduled as a very dangerous drug. Thus, it would be beneficial to deliver psychedelics only if an individual is predicted to be a responder.

Transcranial Magnetic Stimulation (TMS) is a non-invasive medical procedure where strong magnetic fields are utilized to stimulate specific areas of an individual's brain to treat medical disorders such as depression and obsessive-compulsive disorder (OCD). When TMS is repeatedly applied in a short time frame, it is referred to as repetitive TMS (rTMS). Theta-burst stimulation (TBS) is a patterned form of rTMS, typically administered as a triplet of stimulus pulses with 20 ms between each stimulus in the triplet (therefore having a pulse frequency of 50 Hz), where the triplet is repeated every 200 ms (therefore having triplets, or bursts, occurring at a frequency of 5 Hz), although other combinations of pulse and burst timing may also be used. Acute rTMS therapy is an approved and acknowledged treatment for MDD (Perera T, George M S, Grammer G, et al. Brain Stimulat 2016, 9:336-346; Milev R V, Giacobbe P, Kennedy S K, et al. Can J Psychiatry Rev Can Psychiatr 2016, 61:561-575) and has been shown to achieve significant antidepressant effects (Sehatzadeh S, Daskalakis Z J, Yap B, Tu H A, Palimaka S, Bowen J M, O'Reilly D J. J Psychiatry Neurosci, 2019, 44:151-163). Acute rTMS therapy has demonstrated similar response and remission rates compared to antidepressant medication therapy alone (monotherapy) as well as psychotherapy plus antidepressants (Baeken C, Brem A-K, Arns M, et al. Curr Opin Psychiatry, 2019, 32:409-415).

Another form of neurostimulation therapy that may be used to treat psychiatric or neurological disorders is focused ultrasound (fUS). fUS is an emerging, non-invasive therapeutic modality that employs ultrasonic energy to target regions of the body. Akin to TMS, in principle, fUS uses constructive interference to magnify pulses of energy directed to a specific target. When directed to a region of the brain, fUS may be used to depress or stimulate neurons. The mechanism of action here is largely mechanical, however, the thermal effects of fUS may also be used to stimulate or lesion. These thermal effects may be used to identify a target region prior to treatment through the guidance of a thermographic imaging modality such as ultrasound, fMRI, and/or fNIRS.

Although acute therapy may be successful, patients with depression, especially treatment resistant depression (TRD), may have a high relapse rate, with relapse occurring weeks or months after acute TMS treatment. Also, depression may be episodic, and a patient who has responded or remitted after acute TMS treatment may relapse by entering a new episode of depression months or years after acute TMS treatment. In patients who have responded previously to TMS therapy, re-treatment with TMS therapy has been shown to be effective when relapse occurs. However, waiting for relapse is undesirable because the symptoms of depression must be experienced again before re-treatment occurs. Accordingly, it would be beneficial to have a system and method for predicting response to treatment when relapse is predicted or observed.

Maintenance rTMS therapy (that is, re-treatment with rTMS therapy without requiring that relapse has fully occurred) for patients with depression may be undertaken using fixed maintenance schedules (for instance, one session or day of maintenance treatment per month, or one week of sessions per six-month period). The development of maintenance rTMS therapy may effectively reduce or prevent the relapse of depression, and decrease the overall burden of depression symptoms, in depression patients who initially responded to acute rTMS treatment. However, maintenance TMS therapy using a fixed maintenance schedule is not generally used in part due to the cost, inconvenience, and potential exposure to side effects related to overtreatment, such as mania, and undertreatment, such as depression, suicide, job and relationship loss, hospitalization, stigma, and the exacerbation of existing medical conditions.

Additionally, a generalized TMS maintenance protocol may fail to address issues related to population variance. Given the diversity of individuals within the human population, generalized medicine may cause some individuals to receive inequitable care. Personalized medicine attempts to circumvent those issues related to generalized therapeutic treatments by employing methods that assist clinicians in making decisions related to clinical care. Such methods may produce outputs based on input data that includes data features collected from a respective individual.

Accordingly, it would be useful to have systems and methods capable of predicting relapse based on readily available data, for example, data from standard inventories. Clinicians may be more inclined to adopt such systems and methods and initiate treatment when needed given that administration of these inventories fits within their current workflow. The automatic delivery of treatment when relapse is predicted would also be useful.

### SUMMARY

Described herein are systems and methods for predicting response to treatment for a neurological or a psychiatric disorder and/or relapse of a neurological or a psychiatric disorder after treatment. The systems and methods may include machine learning and run LDA (Linear Discriminant Analysis) and/or Lasso (Least Absolute Shrinkage and Selection Operator) Regression protocols that use psychometric inventories and other forms of patient data as inputs to generate a predictive model that separates (e.g., identifies) responders from non-responders. The patient data may include, for example, patient characteristics (e.g., height, weight, race, geographic location of residence, and/or sex), treatment history, clinical history, biometric data, neuroimaging data, psychometric inventories, or a combination thereof. The predictive model may also predict patient response to initial treatment with minimal clinician intervention. Further, the predictive model may predict those individuals who will not respond at the beginning of the initial course of treatment, but who may respond later during the initial course of treatment.

The systems and methods may be used to predict response to therapeutic intervention of various psychiatric disorders such as, but not limited to, depression, treatment-resistant depression, anxiety, post-traumatic stress disorder (PTSD), obsessive-compulsive disorder (OCD), a substance use disorder, bipolar disorder, and schizophrenia. Exemplary neurological disorders in which the systems may be used to predict relapse include without limitation, Parkinson's disease, essential tremor, stroke, epilepsy, traumatic brain injury, migraine headache, cluster headache, and chronic pain.

The systems and methods for predicting response and/or relapse of a neurological or a psychiatric disorder of a patient may generally include a device configured to obtain one or more data features from the patient and a data module. The obtained data features may be transmitted (e.g., wirelessly or by a wired/cabled connection) to the data module. In some variations, the data features may be manually input to the data module using a user interface of the data module. The data module may comprise one or more processors configured to run one or more predictive methods (e.g., one or more processes or protocols). In one variation, the one or more processors may be configured to run one or more LDA methods, where the LDA methods may be configured to analyze the one or more data features, generate a mood plot that separates responders from non-responders based on the analyzed one or more data features, and predict response during initial or subsequent treatment of the neurological or the psychiatric disorder in the patient based on the mood plot. Instead of running one or more LDA methods, in some variations, the one or more processors may be configured to run one or more Lasso Regression methods, where the Lasso Regression methods may be configured to analyze the one or more data features, generate a mood plot that separates responders from non-responders based on the analyzed one or more data features, and predict response during initial or subsequent treatment of the neurological or the psychiatric disorder in the patient based on the mood plot.

The predictive methods may process data from a psychometric inventory obtained from a patient, and based on this data, the patient may be classified into a patient response group. For example, the method may employ LDA to reduce the number of features to a more manageable number before classification into a response group. The evaluated features may be from the administered psychometric inventories. LDA may be used to determine the linear combination of features that best separates classes (e.g., responders vs. non-responders). Alternatively, the predictive method may employ Lasso Regression to eliminate poorly predictive features from the model and may perform feature selection on highly predictive features, as further described herein. The features may be selected from the administered psychometric inventories. The Lasso Regression may then be used to produce a subset of features for building a predictive model. In addition to predicting response, the methods may further include delivering treatment, e.g., neurostimulation such as transcranial magnetic stimulation.

The systems and methods may be designed to issue an alert or other warning signal that notifies the clinician and/or patient that the patient will likely not respond to a proposed treatment during initial or maintenance treatment courses. The alert may be an audible alarm, a visual alarm, a text, an email, or a combination thereof. In some instances, the system may include a treatment device, for example, a transcranial magnetic stimulation (TMS) device or a device (e.g., a transducer) configured to deliver focused ultrasound (fUS), that may be automatically triggered or manually activated to deliver neurostimulation therapy upon receipt of the prediction of response.

The systems and methods described herein may be useful in predicting patient response and may be employed by clinicians to determine a course of treatment for individuals experiencing a neurological and/or a psychiatric disorder. In short, by determining patient data features that best predict response to treatment and by removing those items that are least predictive, the predictive model may be able to group patients into response categories based on data features such as psychometric inventory scores, clinical and treatment data, and biometric data.

Prior to administration of therapy, a clinician may employ a predictive method that uses data from psychometric inventories to predict whether an individual would respond to a treatment, such as TMS. For example, the method may be employed to predict whether an individual will respond, e.g., on day one, two, three, four, and five of the initial course of treatment by grouping an individual as a responder or non-responder. Further, the method may be used to predict whether an individual will continue responding to treatment during weeks one, two, three, four, five, six, seven, eight, nine, and ten (or more) of maintenance treatment. In some variations, the method may be used to predict whether an individual may initially respond to treatment, but not respond during subsequent treatments or maintenance therapy. In other variations, the method may predict whether an individual may not initially respond to treatment, but may respond later during initial treatment or maintenance therapy.

Methods for providing neurostimulation therapy that include predicting a patient's response to treatment are also described herein. The methods may include obtaining data from a patient having a neurological disorder or a psychiatric disorder, and analyzing the data using one or more machine learning methods, where analyzing includes selecting one or more features from the data. The methods may also include generating a report based on the selected one or more features, and determining whether the patient will respond to a neurostimulation treatment based on the report. The report may be a report downloaded and/or printed from a data module of the systems described herein, or a report provided on a display of a user interface of the data module, processor, or other component of the system.

As previously mentioned, the neurological disorder may be Parkinson's disease, essential tremor, stroke, epilepsy, traumatic brain injury, migraine headache, cluster headache, or chronic pain, and the psychiatric disorder may be depression, treatment-resistant depression, anxiety, post-traumatic stress disorder (PTSD), obsessive-compulsive disorder (OCD), a substance use disorder, bipolar disorder, or schizophrenia.

Any suitable machine learning method may be employed. In some variations, the machine learning methods may comprise Linear Discriminant Analysis (LDA) or Lasso Regression. The data used by the machine learning methods may include a characteristic of the patient, a treatment history of the patient, a clinical history of the patient, biometric data, neuroimaging data, or a combination thereof. More specifically, the data may include one or more features for analysis that may be selected from a psychometric inventory such as the Hamilton Depression Rating Scale (HAM-D) and/or the Montgomery-Asberg Depression Rating Scale (MADRS). It is understood that features from other psychometric inventories may also be used. In some variations, the one or more features selected from the psychometric inventory for analysis comprises apparent sadness, reported sadness, inner tension, reduced sleep, reduced appetite, concentration difficulty, lassitude, inability to feel, pessimistic thoughts, and suicidal thoughts. In one variation, it may be beneficial to analyze reduced sleep. In other variations, the one or more features selected from the psychometric inventory for analysis comprises depressed mood, feelings of guilt, suicide, insomnia, effect on work, effect on activities, somatic anxiety, psychic anxiety, somatic gastro-intestinal symptoms, general somatic symptoms, genital symptoms, and weight loss.

Additionally or alternatively, the one or more features for analysis may be selected from a cognitive assessment. An exemplary cognitive assessment is the Creyos cognitive assessment. Features such as feature match, grammatical reasoning, and token search may be useful to analyze from this assessment.

In one variation of the method, the machine learning method may include Linear Discriminant Analysis (LDA) and the one or more features may include reduced sleep, lassitude, retardation, and reduced appetite.

In another variation of the method, the machine learning method may include Lasso Regression and the one or more features may comprise reduced sleep and pessimistic thoughts.

In some variations, the methods may further include selecting a course of treatment for the patient based on the report that is generated. In yet other variations, the methods may further include delivering the neurostimulation treatment to the patient. The neurostimulation treatment may include transcranial magnetic stimulation (e.g., delivered from a magnetic stimulation coil). In some instances, the neurostimulation treatment may be focused ultrasound (fUS) (e.g., delivered from an ultrasound transducer). In further instances, the neurostimulation treatment is delivered using an implantable or partially implantable device. The methods may further include sending an alert to a clinician or the patient when it is determined that the patient will not respond to the neurostimulation treatment. The alert may be provided by email, text message, or an audible sound.

In some variations, the systems for providing neurostimulation therapy and predicting a patient's response to the therapy may include a device configured to obtain data from the patient having a neurological disorder or a psychiatric disorder and a data module comprising one or more processors configured to run one or more machine learning methods, where the one or more machine learning methods analyzes the data from the patient by selecting one or more features from the data. The systems may also include a report generator configured to generate a report based on the selected one or more features. The device may be a computer, a laptop, a tablet computer, a mobile phone, a smart watch, or a smart ring. In some variations, the device may be an implantable device or a partially implantable device. The systems may further comprise a treatment device for delivering neurostimulation treatment. For example, the treatment device may include a coil configured to deliver magnetic stimulation, a transducer configured to deliver focused ultrasound (fUS), or an implantable or partially implantable device.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a table that lists exemplary features that may be reduced by LDA. The exemplary features (scale items) may indicate the items that may classify responders versus non-responders. The column on the left illustrates the exemplary feature (scale item) while the column on the right is the Linear Discriminant (LD) weight assigned to each exemplary feature reduced by LDA.

FIG. 2 is a graph showing whether patients were responders or non-responders based on the weighted features listed in FIG. 1. The shape denotes the clinical research site and the colors denote responder (green) versus non-responder (red).

FIG. 3 is a graph showing whether patients were responders or non-responders based on the top four features listed in FIG. 1. The shape denotes the clinical research site and the colors denote responder (green) versus non-responder (red).

FIG. 4 is a table that lists other exemplary features that may be reduced by LDA. The exemplary features (scale items) may indicate the items that may classify responders versus non-responders. The column on the left illustrates the exemplary feature (scale item) while the column on the right is the Linear Discriminant (LD) weight assigned to each exemplary feature reduced by LDA.

FIG. 5 is a graph showing whether patients were responders or non-responders based on the weighted features listed in FIG. 4. The colors denote responder (green) versus non-responder (red).

FIG. 6 is a table that lists further exemplary features that may be reduced by LDA. The exemplary features (scale items) may indicate the items that may classify responders versus non-responders. The column on the left illustrates the exemplary feature (scale item) while the column on the right is the Linear Discriminant (LD) weight assigned to each exemplary feature reduced by LDA.

FIG. 7 is a graph showing the LDA output for each patient, and whether the patients were responders or non-responders based on the weighted features provided in FIG. 6. The colors denote responder (green) versus non-responder (red).

FIG. 8 is a table that lists exemplary features that may be selected by Lasso Regression and their coefficients. The features (scale items) are presented in the column on the left while the column on the right provides their coefficients. The “.” indicates features with a zero value (which may be weak predictors).

FIG. 9 is a graph showing the Lasso Regression output which may indicate model performance. The lower values may indicate better performance. The dotted vertical line (900) through the lowest red dot may be the best performing model. The features selected for this model and their coefficients are provided in FIG. 8. The other dotted vertical line (902) may be the most penalized model (the fewest features selected) that may perform statistically similar to the best performing model.

### DETAILED DESCRIPTION

Described herein are systems and methods for the prediction of relapse in a patient with a neurological or a psychiatric disorder. The prediction may be based on various data features processed by one or more machine learning methods, and thus may be automated. If relapse is predicted, the systems and methods may be generally configured to trigger re-treatment of the neurological or psychiatric disorder.

The methods and systems for predicting relapse of a neurological or a psychiatric disorder of a patient may generally include a device configured to obtain one or more data features from the patient and a data module. The data module may comprise one or more processors configured to implement one or more machine learning models, where the one or more machine learning models may be configured to analyze the one or more data features, generate a mood report based on the analyzed one or more data features, generate a mood plot having a mood threshold predetermined for the patient based on a plurality of mood reports taken over a plurality of neurostimulation treatment sessions, and predict relapse of the neurological or the psychiatric disorder in the patient based on the mood plot.

There are various data features that may be collected prior to treatment of a neurological or psychiatric disorder. For example, psychometric inventories may be administered prior to and during treatment of a neurological or a psychiatric condition. Given that psychometric inventories are scored quantitatively, they may be analyzed by a predictive method. For example, using Linear Discriminant Analysis (LDA), the predictive method may analyze psychometric inventory data features so that patients may be classified into treatment response groups (e.g., responders vs. non-responders).

In some variations, the psychometric inventory may be the Hamilton Depression Rating Scale (HAM-D). HAM-D is the most widely used clinician-administered depression assessment scale and may also be self-reported. As further described below, the HAM-D is a 17 item, five-point scale that measures features such as depressed mood, feelings of guilt, agitation, and insomnia. The purpose of this inventory is to evaluate the severity of and change in physiological depressive symptoms.

Additionally or alternatively, the Montgomery-Asberg Depression Rating Scale (MADRS) may be used as the psychometric inventory. The MADRS may be administered by clinicians to assess depression. as further described below, the MADRS is a ten item, seven-point scale that measures features such as sadness, inner tension, lassitude, and the inability to feel. The purpose of this inventory is to evaluate the severity of and change in depressive symptoms.

Data features may also be collected prior to treatment of a neurological or psychiatric disorder using cognitive assessments. One example of a cognitive assessment is the Creyos cognitive assessment. The Creyos cognitive assessment is a suite of online neurocognitive tasks that measure core elements of cognition, including memory, attention, reasoning, and verbal ability. Cognitive assessments may be administered prior to or during treatment for a neurological or a psychiatric condition. These assessments are quantitative and thus cognitive data features may be analyzed using a predictive method similar to LDA so that patients may be classified into treatment response groups (e.g., responders vs. non-responders). Other examples of cognitive assessments that may be administered prior to treatment include without limitation, the VoxNeuroCore cognitive assessment and the Mini-Cog and Mini-Mental State Exam (MMSE).

As previously mentioned, the psychiatric disorders that may be treated with the systems and methods disclosed herein include without limitation, depression, treatment-resistant depression, anxiety, post-traumatic stress disorder (PTSD), obsessive compulsive disorder (OCD), addictions, substance use disorders such as opioid, stimulant, tobacco, or alcohol use disorders, bipolar disorder, and schizophrenia. The neurological disorders and associated symptoms that may be treated with the systems and methods disclosed herein include without limitation, Parkinson's disease, essential tremor, stroke, epilepsy, traumatic brain injury, migraine headache, cluster headache, and chronic pain.

### Methods

Various protocols may be used to predict patient responses to treatment. In one variation, LDA may be used. Additionally or alternatively, Lasso Regression may be employed. The LDA and Lasso Regression protocols may allow a clinician to predict response to therapeutic interventions prior to and during neurological treatment, such as TMS. Further, these protocols may help more individuals to be treated with less staff, with less clinical issues due to human error, and with more rapid communication of need for more or less treatment at any time of day.

Linear Discriminant Analysis (LDA) is a method of predictive modeling that may be used to reduce the number of features within one or more psychometric inventories, or other patient data inventory, to a more manageable number before classification. In short, the predictive model may be used to determine the linear combination of features that may be useful in grouping patients into classes (e.g., responders vs. non-responders). For example, the predictive model may use the equation:

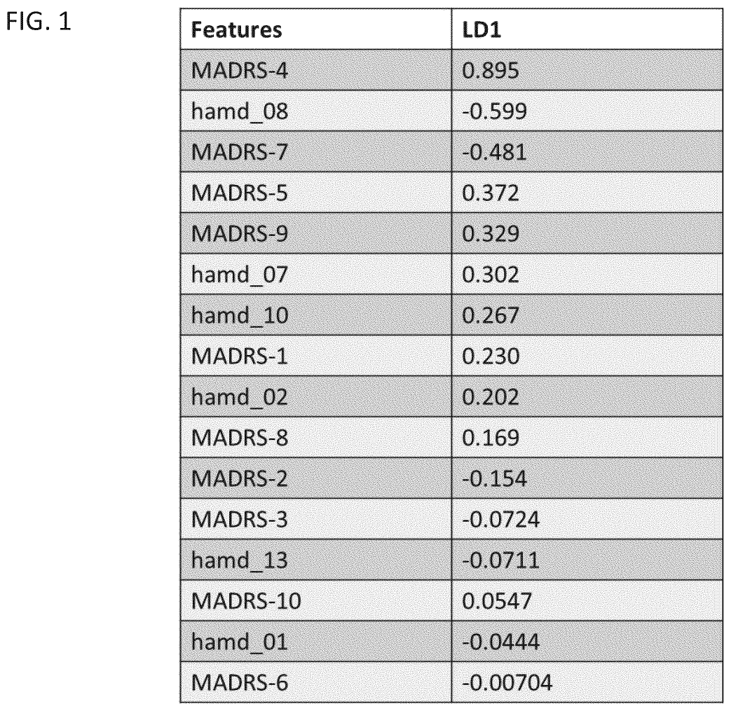

b₁x₁+b₂x₂+ . . . +bₙxₙ

where x is a vector of features and b is a vector of feature weights. This stratification of patient groups may allow clinicians to choose a proper course of treatment. The features to be evaluated may include, but are not limited to, items within one or more psychometric inventories. For example, the evaluated features may be one or more of those listed in FIGS. 1, 4, and 6.

Lasso (Least Absolute Shrinkage and Selection Operator) Regression is a linear regression technique that adds a regularization to the linear regression loss function, which encourages some of the model's coefficients (features) to become exactly zero. This means Lasso Regression may perform feature selection by setting some feature coefficients to zero while keeping others non-zero. Regularization is a statistical technique that is used to avoid overfitting of training data by maintaining the number of features used, constant and by reducing the magnitude of the respective coefficients. When using this technique, the hyperparameter alpha (X) may need to be adjusted to control the amount of regularization. Larger values of alpha may lead to more aggressive feature selection (more coefficients set to zero), while smaller values may allow more features to be retained. After training the Lasso Regression model, features with non-zero coefficients may be considered important and retained, while those coefficients set to zero are excluded from the model. This method of feature selection may be used to select features within one or more psychometric inventories, or other patient data inventory, which may be predictive. The predictive model generated based on the features selected using the Lasso Regression may better inform the clinician about the course of treatment to pursue.

As previously mentioned, predictive methods such as LDA and/or Lasso Regression may be employed to improve a clinician's ability to determine the best course of treatment for an individual. Further, these protocols may increase clinical efficiency and decrease the probability of treatment issues caused by human error. In general, use of LDA and/or Lasso Regression may comprises obtaining data features from the individual who is or will undergo treatment for a neurological or psychiatric disorder. Such data features may include, but are not limited to, the data features described above, data features from psychometric inventories such as the MADRS and HAM-D, bio signals such as skin conductance, heart rate volume, and respiratory rate, biometric data, including genetic data, clinical history (including treatment history), neuroimaging data, or combinations thereof.

In some variations, LDA and/or Lasso Regression may be used to predict response to a therapeutic intervention by using baseline results of psychometric inventories that assess the severity of a neurological or psychiatric condition. The therapeutic intervention may include, but is not limited to, the clinical administration of, ketamine, psychedelics, transcranial magnetic stimulation (TMS), deep brain stimulation, and focused ultrasound. TMS is a non-invasive medical procedure where strong magnetic fields may be utilized to stimulate specific areas of a patient's brain. These therapeutic interventions may be assisted by imaging modalities including, but not limited to, magnetic resonance imaging (“MRI”) by way of example functional MM (“fMRI”) including thermographic variations, diffuse optical imaging (“DOI”), computer-aided tomography (“CAT”), event-related optical signal (“EROS”) imaging, Magnetoencephalography (“MEG”), positron emission tomography (“PET”) by way of example single-photon emission computerized tomography (“SPECT”) imaging, electroencephalography (“EEG”), and/or functional near-infrared spectroscopy (“fNIRS”).

For example, patient data from two study sites (one study including 40 subjects, the other study including 13 subjects) was used to determine if a subset of MADRS and/or HAM-D scores administered at baseline could predict response to TMS treatment for depression. Response was defined by a greater than 50% decrease in total MADRS score.

The MADRS inventory was administered by a clinician. The MADRS inventory may be used to stratify the severity of depression in adults. The MADRS is a 10-item inventory. The MADRS evaluates the following items: apparent sadness (e.g., despondency, gloom, and despair reflected in speech, facial expression, and posture); reported sadness; inner tension (e.g., feelings of ill-defined discomfort, inner turmoil, etc.); reduced sleep; reduced appetite; concentration difficulty; lassitude (e.g., difficulty getting started or slowness initiating and performing everyday tasks); inability to feel (e.g., subjective experience of reduced interest in surroundings or activities that normally give pleasure); pessimistic thoughts; and suicidal thoughts.

The HAM-D was also administered by a clinician. The HAM-D is the most widely used depression assessment scale. The main purpose of the scale is to assess the severity of, and change in, depressive symptoms. The HAM-D evaluates the following items: depressed mood; feelings of guilt; suicide; insomnia: early in the night insomnia; middle of the night insomnia; early hours of the morning insomnia; work and activities; retardation (e.g., slowness of thought and speech, impaired ability to concentrate); agitation; anxiety psychic; anxiety somatic (e.g., physiological concomitants of anxiety); somatic gastro-intestinal symptoms; general somatic symptoms; genital symptoms (e.g., loss of libido, menstrual disturbances); hypochondriasis; weight loss a) according to patient and b) according to weekly measurements; and patient insight regarding their illness.

In these studies, Linear Discriminant Analysis (LDA) was used to reduce the number of features to a more manageable number before classification. Each feature listed in the MADRS and HAM-D inventories were evaluated. LDA was used to determine the linear combination of features (MADRS and HAM-D items) useful for grouping patients into classes (e.g., responders vs. non-responders).

Lasso Regression may be used be used separately or in combination with LDA to select the features that may be useful for predicting relapse. Lasso Regression is a linear regression technique that adds a regularization term to the linear regression loss function, which encourages some of the model's coefficients (features) to become exactly zero. This means Lasso Regression may perform feature selection by setting some feature coefficients to zero while keeping others non-zero. Each feature in the MADRS and HAM-D inventories were evaluated. After training the Lasso Regression model, features with non-zero coefficients were considered predictive and retained, while those with coefficients set to zero were excluded from the model.

For example, the features reduced by LDA in the studies are provided in the table shown in FIG. 1. The features are the items that may be useful for classifying responders versus non-responders. The column on the left lists the features while the column on the right provides the linear discriminant weight assigned to each feature reduced by LDA.

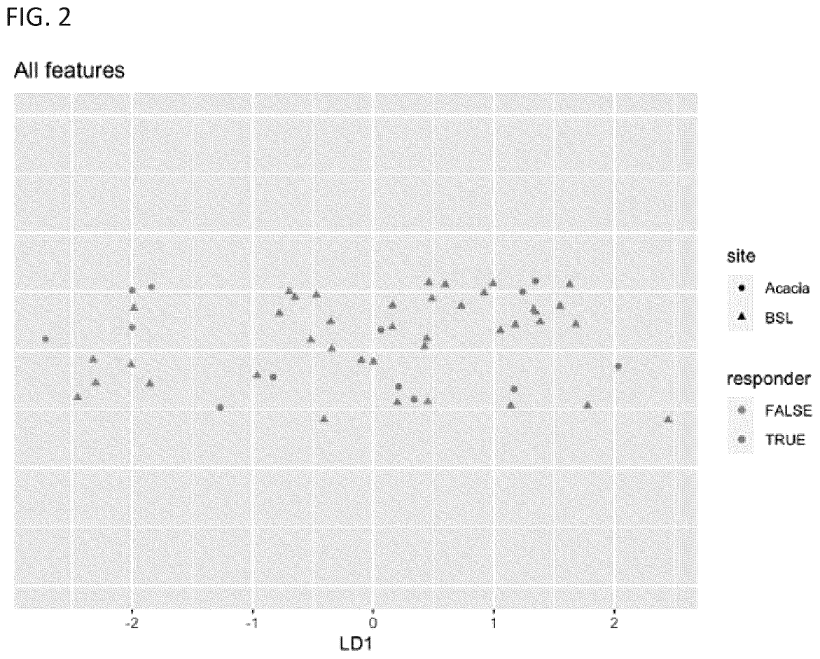

In FIG. 2, the LDA output for each patient based on all the weighted features in FIG. 1 is shown. The shapes denote the respective clinical research site while the colors denote responder (green, true) vs. non-responder (red, false).

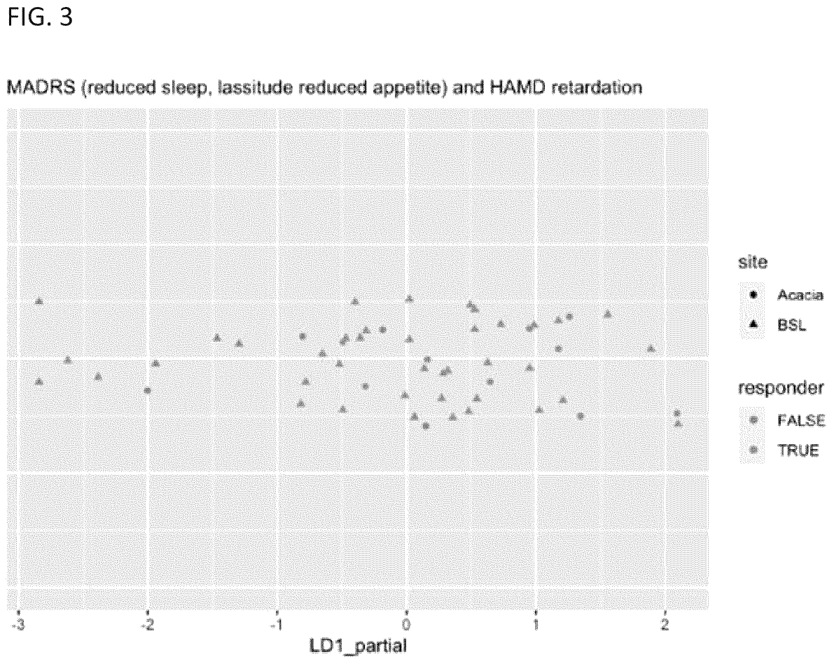

Referring to FIG. 3, the LDA output for each patient based on the top four features in FIG. 1 is illustrated. Based on the LDA output, three MADRS features (reduced sleep, lassitude, and reduced appetite) and one HAM-D feature (retardation) may provide good predictors of response.

Other variations of the method may include model optimization through the inclusion of additional psychometric inventories having features that may be assessed by the LDA method. Examples of such psychometric inventories may include without limitation, the Beck Depression Inventory II (BDI-II), the Patient Health Questionnaire, the Mental Health Quality of Life Questionnaire (MHQoL-7D), Generalized Anxiety Disorder questionnaire (GAD-7), Center for Epidemiologic Studies Depression Scale, the EQ-5D, the Social Problem-Solving Inventory Revised (SPSI-RTM), the Behavior Assessment System for Children (BASC), the Beck Hopelessness Scale, the Quick Inventory of Depressive Symptomatology, and the Social Functioning Questionnaire.

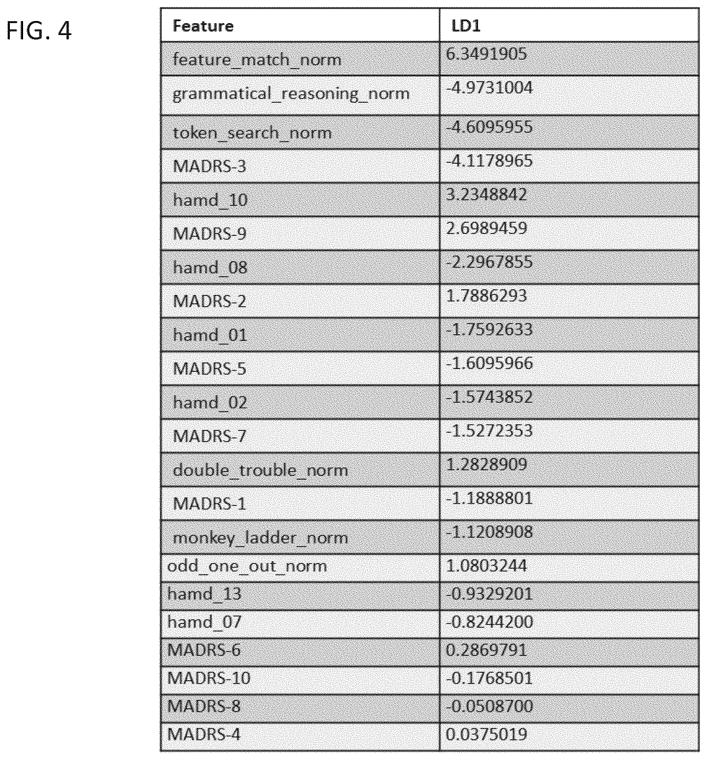

In another variation, LDA may be used to help predict response of a patient to treatment, as illustrated in FIGS. 4-7.

Referring to the table in FIG. 4, the column on the left lists the features that may be reduced by LDA. The features included items from psychometric inventories (MADRS and HAM-D) and a cognitive assessment (Creyos) administered to patients prior to treatment. The listed features may be useful in classifying responders versus non-responders. The column on the left lists the features while the column on the right provides the linear discriminant weight assigned to each feature reduced by LDA. As shown in FIG. 4, the top-weighted features are those from the cognitive assessment (e.g., grammatical reasoning and token search).

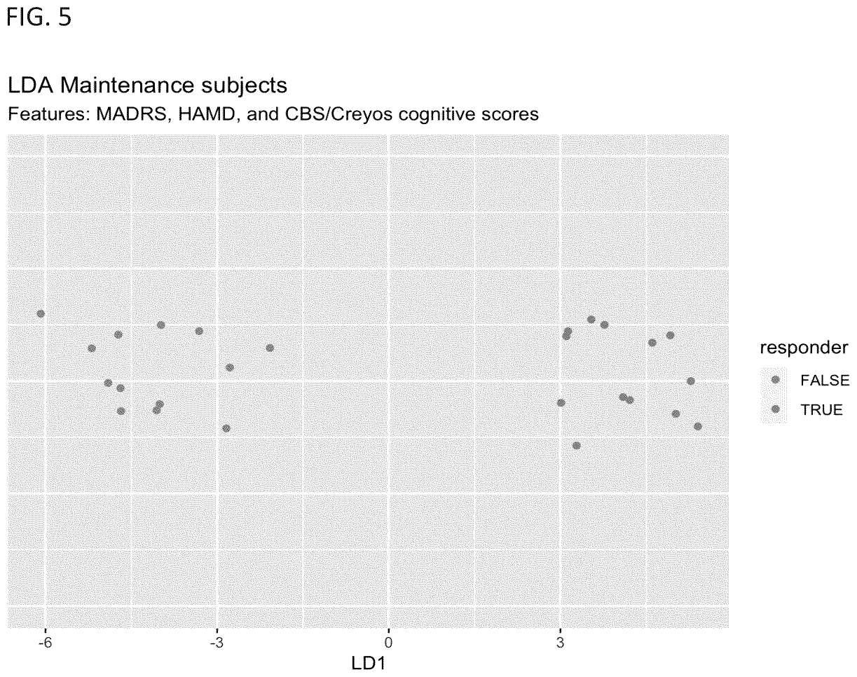

Referring to FIG. 5 the LDA output for each patient based on the weighted features in FIG. 4 is illustrated. The colors denote responder (green, true) vs. non-responder (red, false). The model was able to predict patient response as illustrated by the clear separation between responders and non-responders.

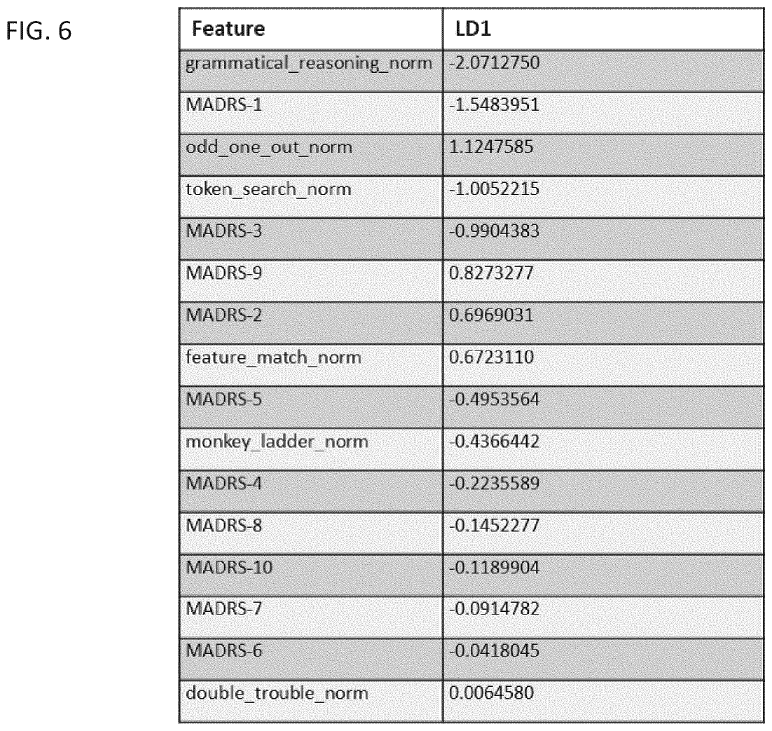

Referring to the table in FIG. 6, the column on the left lists the features that may be reduced by LDA. The features included items from one psychometric inventory (MADRS) and a cognitive assessment (Creyos) administered to patients prior to treatment, e.g., treatment with TMS. The listed features may be useful in classifying responders vs. non-responders. The column on the left lists the plurality of features while the column on the right provides the linear discriminant weight assigned to each feature reduced by LDA. The top-weighted features may again be those from the cognitive assessment (e.g., feature match, grammatical reasoning, and token search).

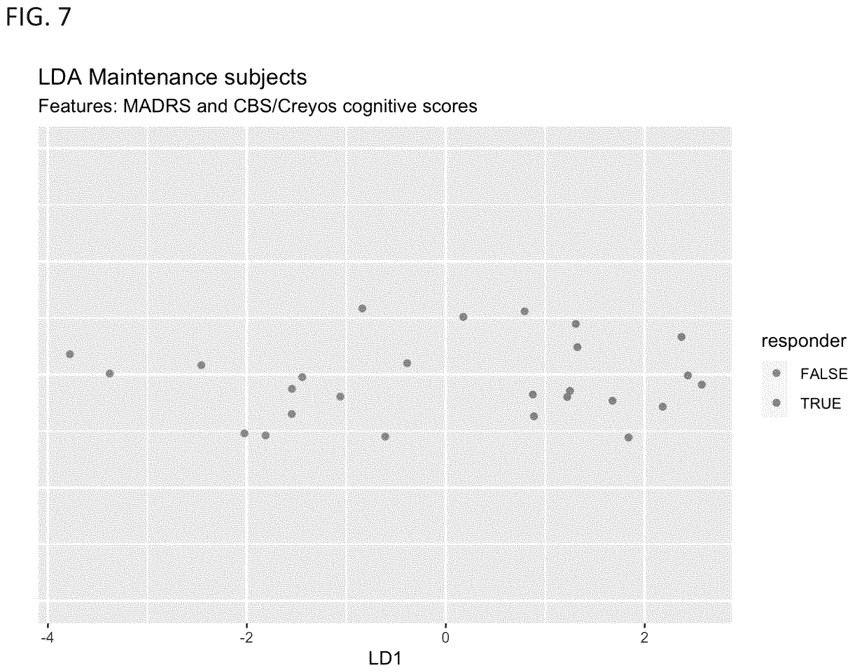

The LDA output for each patient based on the weighted features in FIG. 6 is illustrated in FIG. 7. In FIG. 7, the colors denote responder (green, true) vs. non-responder (red, false). The model was able to predict patient response as depicted by the separation between responders and non-responders.

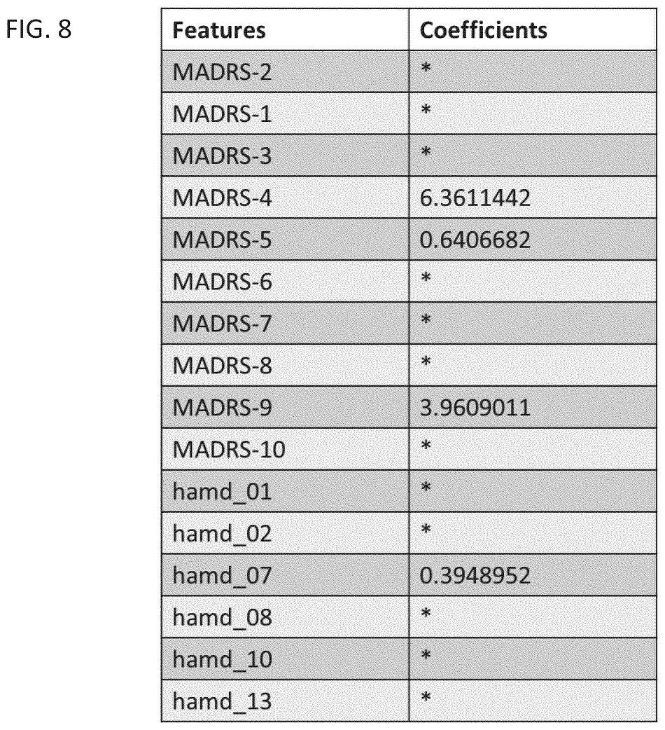

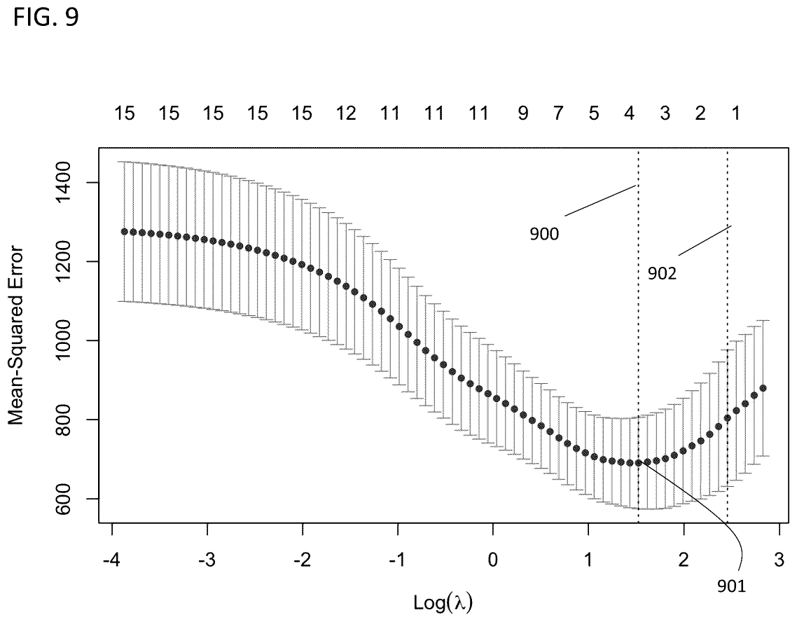

In a further variation, Lasso Regression may be used to help predict response of a patient to treatment, as shown in FIGS. 8 and 9.

Referring to the table in FIG. 8, the plurality of features selected by Lasso Regression to be input into the model and their coefficients are provided. Each feature listed has a corresponding coefficient provided in the column on the right. The “.” indicates features with a zero value (indicating the feature is a weak predictor). Based on the coefficients in FIG. 8, the features that may be useful in predicting response, within this study, may include Reduced Sleep (MADRS 4), Reduced Appetite (MADRS 5), Pessimistic Thoughts (MADRS 9), and Retardation (HAM-D 7). All these selected features have non-zero values. Other features may be selected as useful within the context of a different data set.

The Lasso output may indicate model performance, with lower values generally indicating better performance. For example, referring to the graph provided in FIG. 9, the dotted vertical line (900) through the lowest red dot (901) may be the best performing model. The features selected for this model and their coefficients are shown in FIG. 8. The other dotted vertical line (902) may be the most penalized (regularized) model (the fewest features selected) that performs statistically similar to the best performing model. The x-axis of the graph is the coefficient penalty (i.e., how strongly or not the coefficients are pushed to zero). This axis has been transformed to a log value to make the numbers easier to read (e.g., log(0.05) is approximately −3).

### Systems

The systems described herein generally analyze data and establish models to make predictions, e.g., predictions of response to treatment for a neurological or a psychiatric disorder. The systems may comprise one or more processors operable to run one or more predictive engines (also referred to as “engines”). The one or more predictive engines may include a data processing framework configured to implement one or more methods, e.g., LDA and/or Lasso Regression, trained and configured based on collections of data.

In some variations, the systems described herein may generally include a device configured to obtain one or more data features from the patient and a data module. The device for obtaining the one or more data features may comprise a computer, a laptop, a tablet computer, a mobile phone, a smart watch, a smart ring configured to collect data features, or an implantable device or a partially implantable device such as an implant dedicated to collection of data features or a neurostimulation implant configured to collect data features. The data module may comprise one or more processors configured to run an LDA method, where the LDA method may be configured to analyze one or more data features (e.g., one or more psychometric inventory items, cognitive assessment items, etc.), generate a mood report based on the analyzed one or more data features and predict response to treatment of a neurological or psychiatric disorder in the patient based on the mood report. Response may be predicted if the LDA method determines that patient's condition may likely decrease (e.g., deteriorate) below a predetermined threshold.

In some variations, the LDA method may be a support vector machine trained, e.g., on previous patient data, and may be used to analyze a patient's data and determine whether the patient is: (a) not likely to respond in a period of time (e.g., 24 hours, 5 days); or (b) likely to respond in a period of time. The period of time may range from about 4 hours to about one week, including all values and sub-ranges therein. For example, the time period may be about 4, about 5, about 6, about 7, about 8, about 9, about 10, about 11, about 12, about 13, about 14, about 15, about 16, about 17, about 18, about 19, about 20, about 21, about 22, about 23, or about 24 hours; or about 2, about 3, about 4, about 5, about 6, or about 7 days. The time period may also be less than 4 hours or greater than one week. In some variations, the machine learning method may be used to additionally determine (c) whether a patient will respond after an initial 5-day course of treatment. For example, the time period may be n+5 days where n may equal a period of hours days or weeks.

In other variations, the one or more data features may comprise information related to heart rate, heart rate variability, electroencephalography, electrogastrography, electrogastroenterography, galvanic skin response, sleep, sweat chloride, neuroimaging, patient demographics, outcome data from an acute treatment, outcome data from a prior maintenance treatment, or a combination thereof. The information related to sleep may include a total duration of sleep, a sleep onset time, a sleep offset time, a sleep cycle duration, a number of sleep cycles per night, sleep movements, sleep vocalizations, or a combination thereof. In further variations, the one or more data features may include body temperature or fluctuation in body temperature, such as standard deviation of body temperature. The one or more data features may also include information estimated from a clinician administered inventory, such as, but not limited to, MADRS or HAM-D. In another variation, the one or more data features may comprise information related to neuroimaging data such as, but not limited to, EEG, SPECT, MRI, fMRI, doppler ultrasound, and FNIRS.

The system may be configured to prospectively recommend a treatment schedule to increase the likelihood of response, e.g., based on outcomes from acute treatment (such as timing and magnitude of response or remission), patient characteristics (such as age, weight, gender, genetics), neuroimaging data (such as degree of frontal hypoactivity and/or subgenual cingulate hyperactivity before and/or after treatment), or other data features or combinations of the data features described above.

In some variations, the system may be configured to recommend a preliminary maintenance schedule including, e.g., timing and number of days of treatment, after acute treatment is complete, e.g., using a support vector machine or other classification method operating on data features such as patient characteristics and acute outcome data. The support vector machine or classification method may assign the patient to one of two or more categories, for example: (a) no maintenance schedule needed; (b) maintenance needed once per six months; (c) maintenance needed once per month; or (d) maintenance needed once per week.

The system may include a data collection facility or device such as a computer, a laptop, a tablet computer, a mobile phone, a smart-watch, or a wearable device such as a ring or patch, having a mobile application that collects actigraphy or other data features from the patient and that presents a mood inventory at intervals. In some variations, presenting a mood inventory comprises asking the patient to answer one or more questions regarding mood or other symptoms of depression such as anxiety. These questions may be derived from a psychometric inventory, such as the MADRS or HAM-D.

In additional variations, questions regarding mood may be presented via means other than a screen or display interface, such as by signaling the patient to answer using sound or vibration, and/or by the patient responding by tapping, turning, or otherwise moving the wearable device. The collected data may be transmitted to the system via a wired or wireless connection.

Based on these data and/or other data, such as patient characteristics or outcomes from previous treatment, the system may then periodically (e.g., once per day) reclassify the patient as likely or unlikely to respond in various time intervals, and may notify the patient or a clinician if the patient is classified as likely to respond in a given time interval (e.g., in the next 24 hours). Based on these data, the system may also recommend an adapted maintenance schedule, e.g., a more frequent or less frequent maintenance schedule, personalized medicine. In other variations, the system may be configured to learn or adapt from its experience with one patient to others, or from its experience at one point in time with one patient to a later point in time with the same patient, e.g., using a support vector machine or other method.

The systems described herein may also include a treatment device. An exemplary treatment device may be configured to deliver neurostimulation therapy. In one variation, the treatment device comprises a transcranial magnetic stimulation coil configured to deliver transcranial magnetic stimulation (TMS). Other forms of neuromodulation may also be employed such as focused ultrasound. In some variations, the treatment delivered may include medication, psychotherapy, adjustment or activation of an implantable neurostimulator, or other interventions.

When the systems include a treatment device configured to deliver magnetic neurostimulation therapy, the magnetic stimulation may be accelerated theta-burst stimulation (aTBS), such as accelerated intermittent theta-burst stimulation (aiTBS) or accelerated continuous theta-burst stimulation (acTBS), delivered transcranially according to the SAINT (Stanford Accelerated Intelligent Neuromodulation Therapy) protocol. The SAINT protocol may include applying iTBS pulse trains for multiple sessions per day, for several days. In one variation, the SAINT protocol may include the delivery of neurostimulation for five days. More specifically, the neurostimulation may be delivered for 10 sessions a day, with each session lasting 10 minutes, and an intersession interval (the interval between sessions) of 50 minutes.

The stimulation frequency of the TBS pulses may range from about 20 Hz to about 70 Hz, including all values and sub-ranges therein. For example, the stimulation frequency may be about 20 Hz, about 25 Hz, about 30 Hz, about 35 Hz, about 40 Hz, about 45 Hz, about 50 Hz, about 55 Hz, about 60 Hz, about 65 Hz, or about 70 Hz. When iTBS is used, the burst frequency (that is, the reciprocal of the period of bursting, for example if a burst occurs every 200 ms the burst frequency is 5 Hz) of the iTBS pulses may range from about 3 Hz to about 7 Hz, including all values and sub-ranges therein. For example, the burst frequency may be about 3 Hz, about 4 Hz, about 5 Hz, about 6 Hz, or about 7 Hz.

The patient may undergo multiple treatment sessions per day. In some variations, the number of treatment sessions per day may range from 2 sessions to 40 sessions. For example, the number of treatment sessions may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. The number of sessions for iTBS may range from 3 to 15 sessions per day. When cTBS is employed, the number of sessions may range from 10-40 sessions per day. The sessions may be performed on consecutive or non-consecutive days.

Additionally, the duration of the intersession interval may vary and range from about 25 minutes to about 120 minutes, including all values and sub-ranges therein. For example, the intersession interval may be about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes.

In variations using iTBS, the pulse parameters may include 3-pulse trains with 50 Hz pulses at a burst frequency of 5 Hz for 2 second trains, with trains every 10 seconds for 10 minute sessions (1,800 total pulses per session). In some variations, the iTBS schedule may include conducting 10 sessions per day with 50 minute intersession intervals for 5 consecutive days (18,000 pulses per day, and 90,000 total pulses).

When cTBS is used, pulse trains may range from about 4 seconds to about 45 seconds, including all values and sub-ranges therein. For example, the pulse train may be about 4 seconds, about 5 seconds, about 10 seconds, about 15 seconds, about 20 seconds, about 25 seconds, about 30 seconds, about 35 seconds, about 40 seconds, or about 45 seconds. In one cTBS variation, the pulse parameters may include 3-pulse trains with 50 Hz pulses at a burst frequency of 5 Hz for 40 second sessions (600 total pulses per session). In another variation, the cTBS pulse parameters may include 3-pulse trains with 30 Hz pulses at a burst frequency of 6 Hz for 44 second sessions (800 total pulses per session). In many cTBS variations, 30 sessions may be applied per day with 15-minute intersession intervals for 5 consecutive days (18,000 pulses per day, 90,000 total pulses).

It is understood that the pulse parameters and schedules used for maintenance treatment may be varied. For example, the number of pulses or frequency of sessions may be increased or decreased depending on response to treatment of the neurological or psychiatric disorder.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed. The embodiments were chosen and described in order to explain the principles of the invention and its practical applications, they thereby enable others skilled in the art to utilize the invention and various embodiments with various modifications as are suited to the particular use contemplated.

1. A method for providing neurostimulation therapy and predicting response of a patient thereto, comprising:
obtaining data from the patient having a neurological disorder or a psychiatric disorder;
analyzing the data using one or more machine learning methods, wherein analyzing includes selecting one or more features from the data;
generating a report based on the selected one or more features; and
determining whether the patient will respond to a neurostimulation treatment based on the report.

2. The method of claim 1, wherein the neurological disorder is Parkinson's disease, essential tremor, stroke, epilepsy, traumatic brain injury, migraine headache, cluster headache, or chronic pain.

3. The method of claim 1, wherein the psychiatric disorder is depression, treatment-resistant depression, anxiety, post-traumatic stress disorder (PTSD), obsessive-compulsive disorder (OCD), a substance use disorder, bipolar disorder, or schizophrenia.

4. The method of claim 1, wherein the one or more machine learning methods comprises Linear Discriminant Analysis (LDA).

5. The method of claim 1, wherein the one or more machine learning methods comprises Lasso Regression.

6. The method of claim 1, wherein obtaining data comprises acquiring data about a characteristic of the patient, a treatment history of the patient, a clinical history of the patient, biometric data, neuroimaging data, or a combination thereof.

7. The method of claim 1, wherein the one or more features are selected from a psychometric inventory.

8. The method of claim 7, wherein the psychometric inventory is the Hamilton Depression Rating Scale (HAM-D).

9. The method of claim 7, wherein the psychometric inventory is the Montgomery-Asberg Depression Rating Scale (MADRS).

10. The method of claim 7, wherein the one or more features selected from the psychometric inventory comprises apparent sadness, reported sadness, inner tension, reduced sleep, reduced appetite, concentration difficulty, lassitude, inability to feel, pessimistic thoughts, and suicidal thoughts.

11. The method of claim 10, wherein the one or more features comprises reduced sleep.

12. The method of claim 1, wherein the one or more features selected from the psychometric inventory comprises depressed mood, feelings of guilt, suicide, insomnia, effect on work, effect on activities, somatic anxiety, psychic anxiety, somatic gastro-intestinal symptoms, general somatic symptoms, genital symptoms, and weight loss.

13. The method of claim 1, wherein the one or more features are selected from a cognitive assessment.

14. The method of claim 13, wherein the cognitive assessment is the Creyos cognitive assessment.

15. The method of claim 13, wherein the one or more features comprises feature match, grammatical reasoning, and token search.

16. The method of claim 1, wherein the machine learning method is Linear Discriminant Analysis (LDA) and the one or more features comprises reduced sleep, lassitude, retardation, and reduced appetite.

17. The method of claim 1, wherein the machine learning method is Lasso Regression and the one or more features comprises reduced sleep and pessimistic thoughts.

18. The method of claim 1, further comprising selecting a course of treatment for the patient based on the report.

19. The method of claim 1, further comprising delivering the neurostimulation treatment to the patient.

20. The method of claim 1, further comprising sending an alert to a clinician when it is determined that the patient will not respond to the neurostimulation treatment.

21. The method of claim 20, wherein the alert is provided by email, text message, or an audible sound.

22. The method of claim 1, further comprising sending an alert to the patient when it is determined that the patient will not respond to the neurostimulation treatment.

23. The method of claim 22, wherein the alert is provided by email, text message, or an audible sound.

24. A system for providing neurostimulation therapy and predicting response of a patient thereto, comprising:
a device configured to obtain data from the patient having a neurological disorder or a psychiatric disorder;
a data module comprising one or more processors configured to run one or more machine learning methods, wherein the one or more machine learning methods analyzes the data from the patient by selecting one or more features from the data; and
a report generator configured to generate a report based on the selected one or more features.

25. The system of claim 24, wherein the device is a computer, a laptop, a tablet computer, a mobile phone, a smart watch, or a smart ring.

26. The system of claim 24, wherein the device is an implantable device or a partially implantable device.

27. The system of claim 24, further comprising a treatment device.

28. The system of claim 27, wherein the treatment device comprises a magnetic stimulation coil.

29. The method of claim 24, wherein the neurological disorder is Parkinson's disease, essential tremor, stroke, epilepsy, traumatic brain injury, migraine headache, cluster headache, or chronic pain.

30. The method of claim 24, wherein the psychiatric disorder is depression, treatment-resistant depression, anxiety, post-traumatic stress disorder (PTSD), obsessive-compulsive disorder (OCD), a substance use disorder, bipolar disorder, or schizophrenia.

31. The method of claim 24, wherein the one or more machine learning methods comprises Linear Discriminant Analysis (LDA).

32. The method of claim 24, wherein the one or more machine learning methods comprises Lasso Regression.
